(19) United States
(12) Patent Application Publication
(10) Pub. No.: US 2024/0269427 A1
(43) Pub. Date: Aug. 15, 2024

(54) Urinary Catheter Container

(71) Applicant: C. R. Bard, Inc., Franklin Lakes, NJ (US)

(72) Inventors: Ronald N. Legaspi, Alpharetta, GA (US); James David Hughett, Sr., Monroe, GA (US)

(21) Appl. No.: 18/569,470

(22) PCT Filed: Jun. 13, 2022
(86) PCT No.: PCT/US22/33271

§ 371 Date: Dec. 12, 2023

Related U.S. Application Data
(60) Provisional application No. 63/210,398, filed on Jun. 14, 2021.

(51) Int. Cl. A61M 25/00

(52) U.S. Cl. CPC A61M 25/002; A61M 25/0017; A61M 2210/1096

(57) ABSTRACT

Disclosed herein is a urinary catheter assembly including a package container that is transitionable between an expanded open configuration and collapsed closed configuration having the catheter and a bag enclosed therein. The container is transitioned from the closed configuration to the open configuration in one motion by the user pulling tabs in opposite directions. The container may be sterilized along with the catheter, may define a sterile barrier and may include a tamper evident mechanism. The container is securable in the closed configuration enclosing the catheter therein before and after use of the catheter. The catheter assembly may also include a urine collection bag enclosed within the container.

### PRIORITY

This application claims the benefit of priority to U.S. Provisional Application No. 63/210,398, filed Jun. 14, 2021, which is incorporated by reference in its entirety into this application.

### BACKGROUND

Accessing a urinary catheter and fluid collecting bag within packaging material can be difficult for urinary catheter users of low dexterity and low motor skills. It would be beneficially to make it easier for urinary catheter users of low dexterity and low motor skills to more access urinary catheters for use Disclosed herein is a urinary catheter container to addresses the forgoing.

### SUMMARY

Disclosed herein is medical package that, according to some embodiments, includes a container configured to transition from a first closed configuration to an open configuration. The container includes a bottom panel defining a length and a width of the container. The container further includes (i) first and second side panels that are pivotably coupled with the bottom panel along opposite sides of the bottom panel and (ii) first and second end panels that are pivotably coupled with the bottom panel along opposite ends of the bottom panel, where each of the first and second end panels are further coupled with each of the first and second side panels. In the first closed configuration, the first and second end panels and the first and second side panels are disposed substantially parallel to the bottom panel, and the container is configured to enclose a urinary catheter therein in the first closed configuration. In some embodiments, the urinary catheter is a male urinary catheter.

In some embodiments, in the open configuration, the first and second end panels and the first and second side panels are disposed substantially perpendicular to the bottom panel, and in some embodiments, the container is biased away from the first closed configuration toward the open configuration.

In some embodiments, the container is configured for handling within a clean room. In some embodiments, the container is configured to enable sterilization of the catheter when the catheter is disposed within the container, where the container is disposed in the first closed configuration. In some embodiments, the container defines a sterile barrier for the catheter in the first closed configuration.

In some embodiments, the container includes a number of adhesively bonded sections of one or more of the first and second side panels or first and second end panels, where the adhesively bonded sections are configured to secure the container in the first closed configuration. In some embodiments, the number of adhesively bonded sections are configured to define a sealed cavity for containing the urinary catheter therein in the first closed configuration.

In some embodiments, the container is configured to transition from the first closed configuration to the open configuration in response to a user applying a first pulling force to the first side panel and applying a second pulling force to the second side panel, where the second pulling force is directed in opposition to the first pulling force. In some embodiments, the first side panel includes a first pull tab configured for grasping by the user when applying the first pulling force, and the second side panel includes a second pull tab configured for grasping by the user when applying the second pulling force. In some embodiments, at least one of the first and second pull tabs includes a pull ring. In some embodiments, at least a portion of each of the first and second pull tabs extends outward beyond a perimeter of the container in the first closed configuration.

In some embodiments, the urinary catheter includes a bag for collecting urine therein, and in some embodiments, the bag is coupled with the urinary catheter when the bag is disposed within the container, such that the bag is in fluid communication with a lumen the urinary catheter.

In some embodiments, the container is configured to transition from the opened configuration to a second closed configuration, and enclose the urinary catheter within the container in the second closed configuration after use of the urinary catheter. In some embodiments, one or more of the number of adhesively bonded sections are configured to further secure the container in the second closed configuration. In some embodiments, the container is configured to inhibit fluid disposed within the container from exiting the container when the container is disposed in the second closed configuration. In some embodiments, the bottom panel, the first and second side panels, and the first and second end panels each include a hydrophobic inside surface.

In some embodiments, the package further includes a tamper evident mechanism configured to indicate that the container has been transitioned away from the first closed configuration. In some embodiments, the tamper evident mechanism includes a tear-able adhesive tape extending between the first and second side panels.

Also disclosed herein is a urinary catheter assembly that includes a urinary catheter enclosed within any of the embodiments of the package summarized above. In some embodiments, the assembly further includes a collection bag enclosed within the package and in some embodiments, the collection bag is coupled with the catheter such that the bag is in fluid communication with a lumen of the urinary catheter.

Also disclosed herein is a method of draining urine from a bladder that according to some embodiments, includes providing a urinary catheter enclosed within a container in a first closed configuration of the container, where the container includes: a bottom panel defining a length and a width of the container; where the first and second side panels are pivotably coupled with the bottom panel along opposite sides of the bottom panel; where the first and second end panels are pivotably coupled with the bottom panel along opposite ends of the bottom panel; and further where each of the first and second end panels are further coupled with each of the first and second side panels. In the first closed configuration, the first and second end panels and the first and second side panels are disposed substantially parallel to the bottom panel. The method further includes: (i) transitioning the container from the first closed configuration to an open configuration, such that the first and second end panels and the first and second side panels are disposed substantially perpendicular to the bottom panel; (ii) removing the urinary catheter from the container; (iii) utilizing the urinary catheter to drain the urine from the bladder; (iv) placing the urinary catheter back into the container; and (v) transitioning the container from the open configuration to a second closed configuration, such that the first and second end panels and the first and second side panels are again disposed substantially parallel to the bottom panel.

In some embodiments of the method, the container is biased away from the first closed configuration toward the open configuration.

In some embodiments of the method, transitioning the container to the open configuration includes breaching a sterile barrier of the container.

In some embodiments of the method, transitioning the container to the open configuration includes separating a number of adhesively bonded sections of one or more of the first and second side panels or the first and second end panels, the adhesively bonded sections configured to secure the container in the first closed configuration.

In some embodiments of the method, transitioning the container to the second closed configuration includes recoupling one or more of the number of adhesively bonded sections together to secure the container in the second closed configuration.

In some embodiments of the method, transitioning the container to the open configuration includes applying a first pulling force to the first side panel and applying a second pulling force to the second side panel, where the second pulling force is directed in opposition to the first pulling force. In some embodiments of the method, applying a first pulling force to the first side panel and applying the second pulling force to the second side panel includes grasping a first pull tab coupled with the first side panel and grasping a second pull tab coupled with the second side panel.

In some embodiments of the method, the urinary catheter includes a bag for containing urine therein, and the method further includes collecting the urine within the bag; emptying the urine from the bag into a urine receptacle; and placing the bag back into the container.

In some embodiments of the method, the container is configured to inhibit fluid disposed within the container from exiting the container when the container is disposed in the second closed configuration.

In some embodiments of the method, transitioning the container to the open configuration includes tearing an adhesive tape extending between the first and second side panels, where the adhesive tape is configured to provide indication that the container has been transitioned away from the first closed configuration.

In some embodiments of the method, the bottom panel, the first and second side panels, and the first and second end panels each include a hydrophobic inside surface.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A illustrates a perspective view of a container for a urinary catheter and fluid drainage bag in an opened configuration, in accordance with some embodiments.

FIG. 1B illustrates a perspective view of the container of FIG. 1A in a closed configuration, in accordance with some embodiments.

FIG. 2A illustrates another perspective view of the container of FIGS. 1A, 1B the closed configuration further including pull tabs, in accordance with some embodiments.

FIG. 2B illustrates a perspective view of the container of FIG. 2A in the opened configuration, in accordance with some embodiments.

FIGS. 3A-3C illustrate plan views of the container in various stages of transitioning between the opened configuration and the closed configuration, in accordance with some embodiments.

FIG. 4 illustrates a flow chart of an exemplary method of packaging a urinary catheter and fluid drainage bag, in accordance with some embodiments.

FIG. 5 illustrates a flow chart of an exemplary method draining urine from a bladder, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Unless defined otherwise, all technical and scientific terms used herein have the same meaning as commonly understood by those of ordinary skill in the art.

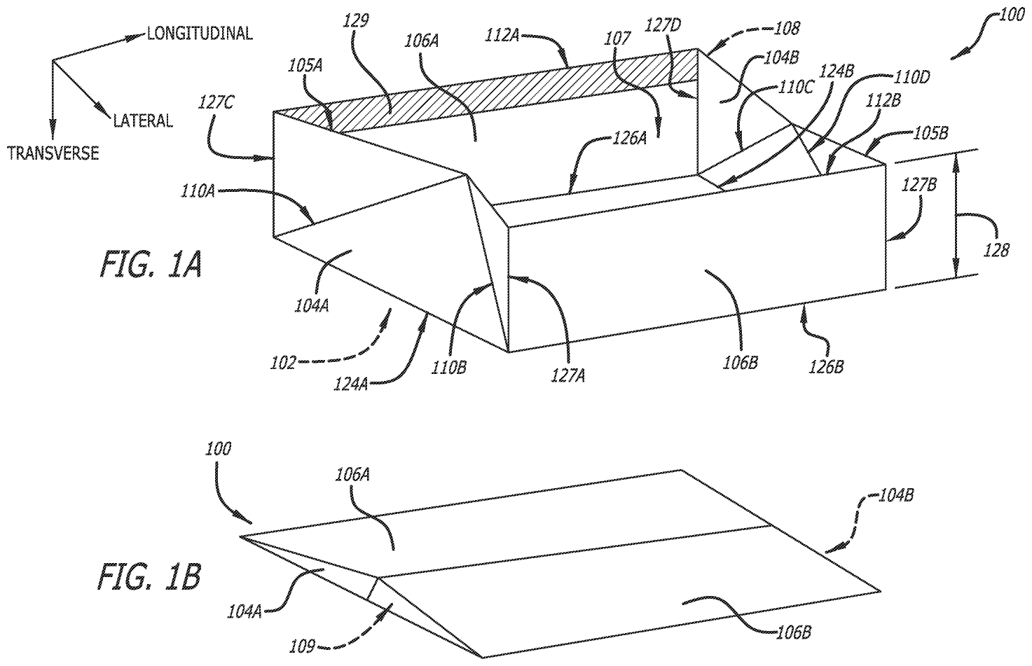

FIG. 1A illustrates a perspective view of a collapsible container (container) 100 in an opened configuration, in accordance with some embodiments. The container 100 is configured to enclose a catheter (e.g., a urinary catheter) therein. The container 100 is configured to transition between a collapsible closed configuration and an expanded opened configuration, the opened configuration enabling a user to retrieve the catheter from the container 100 and return the catheter thereto. The container 100 may also be configured to enclose a drainage bag or urine collection bag therein along with the catheter. The container 100 may define a sterile barrier for the catheter, so that the catheter remains sterile when enclosed within the container 100 before use. In some embodiments, the catheter may include a male urinary catheter. In some embodiments, a shape the container 100 may include rectangle as illustrated or any other polygonal shape. In some embodiments, the container 100 includes a number of walls or panels. In the illustrated embodiment, the container 100 includes a bottom wall 102 defining the shape of the container 100. A first side wall 106A and a second side wall 106B are coupled with the bottom wall 102 along opposite sides of the bottom wall 102. The first side wall 106A and a second side wall 106B are pivotably coupled to bottom wall via creases 126A, 126B, respectively. Similarly, a first end wall 104A and a second end wall 104B are pivotably coupled to the bottom wall 102 via creases 124A, 124B, respectively. The first side wall 106A and a second side wall 106B are further coupled to the end wall 104A via creases 127A, 127C. The first side wall 106A and a second side wall 106B are further coupled to the end wall 104B via creases 127B, 127D.

The first side wall 106A and a second side wall 106B are configured to pivot inward about their respective creases 126A, 126B during transitioning of the container 100 from the open configuration of FIG. 1A to the closed configuration of FIG. 1B. In cooperation with the side walls 106A, 106B, the end walls 104A, 104B are also configured to the pivot inward about their creases 124A, 124B during transitioning of the container 100 from the open configuration the closed configuration. In the opened configuration, the bottom wall 102, the two end walls 104A, 104B and the two side walls 106A, 106B define a cavity 107 having a cavity opening 108, where the cavity opening 108 is configured to allow the user to retrieve the catheter from within the cavity 107. The first side wall 106A and the second side wall 106B include top edges 112A, 112B, respectively. The first end wall 104A and the second end wall 104B include angle creases 110A, 110B and 110C, 110D, respectively to allow cooperation of the end walls 104A, 104B with the side walls 106A, 106B as they each pivot inward. The illustrated embodiment, as shown and described, includes the two end walls 104A, 104B and two side walls 106A, 106B, however, it will be appreciated that a different number of walls defining different arrangements of the walls are also contemplated.

In the illustrated embodiment, the side walls 106A, 106B and the end walls 104A, 104B may be oriented substantially perpendicular to the bottom wall 102 in the open configuration. However, in other embodiments, the side walls 106A, 106B and the end walls 104A, 104B may be oriented at an angle other than 90° with respect to the bottom wall 102 such as an angle within the range of 45-135° in relation to the bottom wall 102, for example.

The container 100 may, according to some embodiments, include a number of adhesive sections of the side walls 106A, 106B and/or the end walls 104A, 104B to cause portions of the side walls 106A, 106B to adhere to corresponding portions of the end walls 104A, 104B while also providing for separation of the adhesive sections. In some embodiments, the adhesive sections may be configured to cause portions of the side walls 106A, 106B to re-adhere (i.e., adhere again after separation) to corresponding portions of the end walls 104A, 104B. For example, the side wall 106A may include an adhesive section 129 disposed on an inside surface the side wall 106A, where the adhesive section 129 is configured to adhere to an inside surface of the end walls 104A, 104B when the container 100 is disposed in the closed configuration. Similar to the exemplary adhesive section 129, the side wall 106B and/or end walls 104A, 104B may include adhesive sections (not shown) on their inside surfaces to cause the side wall 106B to adhere to the end walls 104A, 104B. The end walls 104A, 104B may also include adhesive section on their outside surfaces to cause sections of the end walls 104A, 104B to adhere to themselves when the container 100 is disposed in the closed configuration. The adhesive sections may be configured to secure the container 100 in the closed configuration.

In some embodiments, the container 100 may be biased toward the open configuration so that the container 100 stays in the open configuration to enable to the user to easily remove the catheter from the container 100 and/or place the catheter back into the container 100 after use of the catheter. As such, adhesive sections may secure the container 100 in the closed configuration in opposition to the bias.

The adhesive sections may also be configured define a seal between portions of the side walls 106A, 106B and/or the end walls 104A, 104B to seal the catheter, or other contents such as a fluid (e.g., residual urine), for example, within the container 100 when the container 100 is in the closed configuration. By way of summary, the container 100 may optionally include a number of adhesive sections to (i) secure the container 100 in the closed configuration, and (ii) allow transitioning of the container 100 away from the closed configuration to the open configuration. The adhesive sections may also be configured to re-secure the container 100 in the closed configuration after being disposed in the open configuration. The adhesive sections may also be configured to define a sealed enclosure for the catheter when the container 100 is disposed in the closed configuration.

FIG. 1B illustrates a perspective view of the container 100 in the closed configuration, in accordance with some embodiments. In the closed configuration, the end walls 104A-104B are pivoted inward along their respective creases 124A, 124B so that the end walls 104A-104B are disposed substantially parallel with the bottom wall 102. In cooperation with end walls 104A-104B, the side walls 106A-106B are also pivoted inward along their respective creases 126A, 126B so that the side walls 106A-106B are also disposed substantially parallel with the bottom wall 102 overlapping the end walls 104A-104B. As such, the side walls 106A-106B and the end walls 104A-104B may effectively form a top wall of the container 100 in the closed configuration. The side walls 106A-106B and the end walls 104A-104B together with the bottom wall 102 define a closed cavity 109 that is smaller than the open cavity 107. In the closed configuration, each of the end walls 104A-104B may folded over onto themselves along the creases 110A, 110B and 110C, 110D, respectively.

In some embodiments, the side walls 106A, 106B may include a height 128 that is substantially equal to one half of the width of the bottom wall 102. As such that the top edges 112A, 112B may be disposed adjacent each other when the container 100 is disposed in the closed configuration. In some embodiments, one or both of the side walls 106A, 106B may define a height 128 that is greater than one half of the width of the bottom wall 102 so that the side walls 106A, 106B may partially overlap each other when the container 100 is disposed in the closed configuration.

As shown and discussed, the container 100 is configured to transition between the open configuration and the closed configuration via one fluid movement of moving the side walls 106A, 106B in cooperation with the ends wall 104A, 104B. Advantageously, this one fluid movement allows users of low dexterity and low motor skills to easily access the catheter and the fluid drainage bag for use.

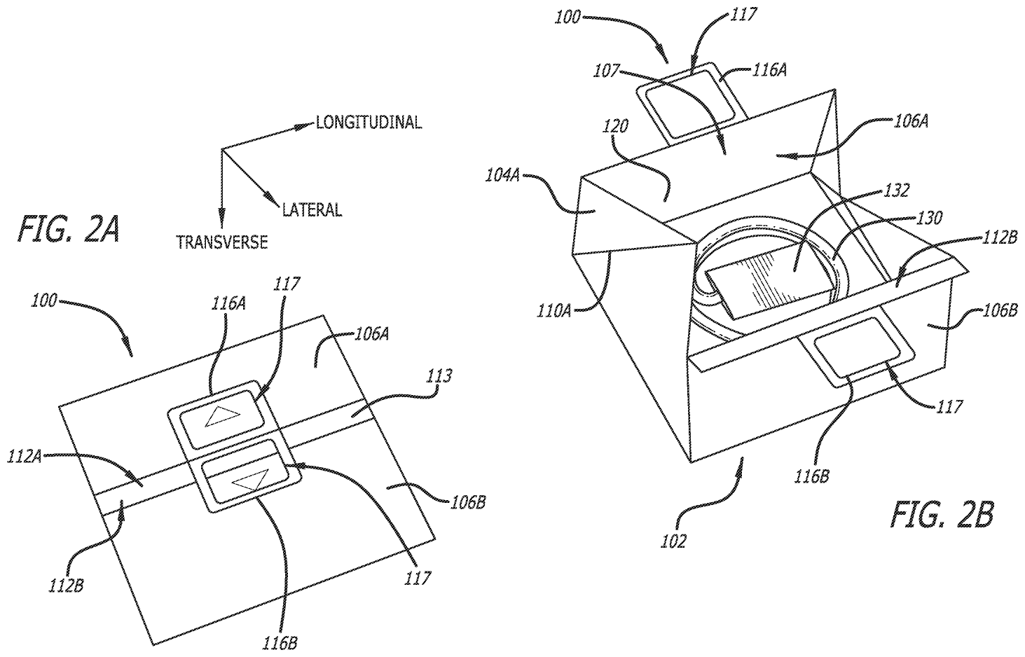

FIG. 2A illustrates a perspective view of the container 100 including further components and features of the container 100, in accordance with some embodiments. In some embodiments, the container 100 may include a number (e.g., 1, 2, 3, 4 or more) of pull tabs 116A, 116B, configured to help the user transition the container 100 from the closed configuration to the opened configuration. In some embodiments, the pull tabs 116A, 116B may be coupled with the side walls 106A, 106B. In some embodiments, the pull tabs 116A, 116B may be formed integrally from the side walls 106A, 106B, stamped from the side walls 106A, 106B, or may be extensions of the side walls 106A, 106B. In some embodiments, the pull tabs 116A, 116B may each include a pull ring 117. During use, the user may transition the container 100 from the closed configuration to the open configuration by applying a first pulling force to the first side wall 106A and a second pulling force to the second side wall 106B, where the second force is directed in opposition to the first pulling force. In some embodiments, the user may grasp the first pull tab 116A when applying the first pulling force and may grasp the second pull tab 116B when applying the second pulling force.

In some embodiments, the container 100 may be secured in the closed configuration by coupling the first side wall 106A to the second side wall 106B. For example, the top edges 112A, 112B of the side walls 106A, 106B may be coupled to each other via an adhesive tape 113 as illustrated in FIG. 2A. In some embodiments, the adhesive tape 113 may define a tamper evidence mechanism to indicate that the container 100 has been transitioned away from the closed configuration. In other embodiments, the side walls 106A, 106B may be coupled to each other via an adhesive between overlapping portions of the side walls 106A, 106B adjacent the top edges 112A, 112B. In some embodiments, the first side wall 106A and the second side wall 106B may be formed integrally with a tear line dividing the first side wall 106A from the second side wall 106B, wherein the tear line is configured to provide a line of weakness configured to allow the side walls 106A, 106B to be separated when the first and second pulling forces are applied thereto. The tear line can include a score line, a groove, a laser cut line, a perforation, a thinned portion or similar structure to facilitate separation therealong. In an embodiment, the one or more pull tabs 116 may be defined by a tear line, allowing a user to separate a portion of each either or both pull tabs 116A, 116B from the side walls 106A, 106B, such as a portion including the pull ring 117.

The container 100 may be formed from a single monolithic piece or may be formed from multiple pieces coupled together. In some embodiments, the container 100 may be formed from polymer, plastic, cardboard, paper, a combination thereof, or the like. In some embodiments, the container 100 may be disposable, reusable, or recyclable. In some embodiments, the container 100 may be configured so as to be compatible with placement and handling within a clean room, so that the catheter 130 and or the drainage bag 132 may be assembled and placed within the container 100 at the same manufacturing location, e.g., within the same clean room. In some embodiments, the container 100 may be formed of a material compatible with sterilization processes, such as radiation, steam, chemicals, or ultraviolet light, for example. In some embodiments, the catheter 130 and the container 100 may be sterilized via the same process as the same time. In some embodiments, the container 100 may be formed of a hydrophobic material or may include a hydrophobic coating across at least inside surfaces of the container 100.

FIG. 2B illustrates a perspective view of the container 100 in the opened configuration, in accordance with some embodiments. As discussed above, the container 100 includes a cavity inner surface 120 which may be hydrophobic. In some embodiments, the cavity inner surface 120 may include features, e.g., indentations, protrusions, ridges, or flaps, for example, to help stabilize the catheter 130 and/or the fluid drainage bag (or urine collection bag) 132 within the container 100 during transport. In some embodiments, the features may be on the side walls 106A, 106B, the end walls 104A, 104B, or the bottom wall 102.

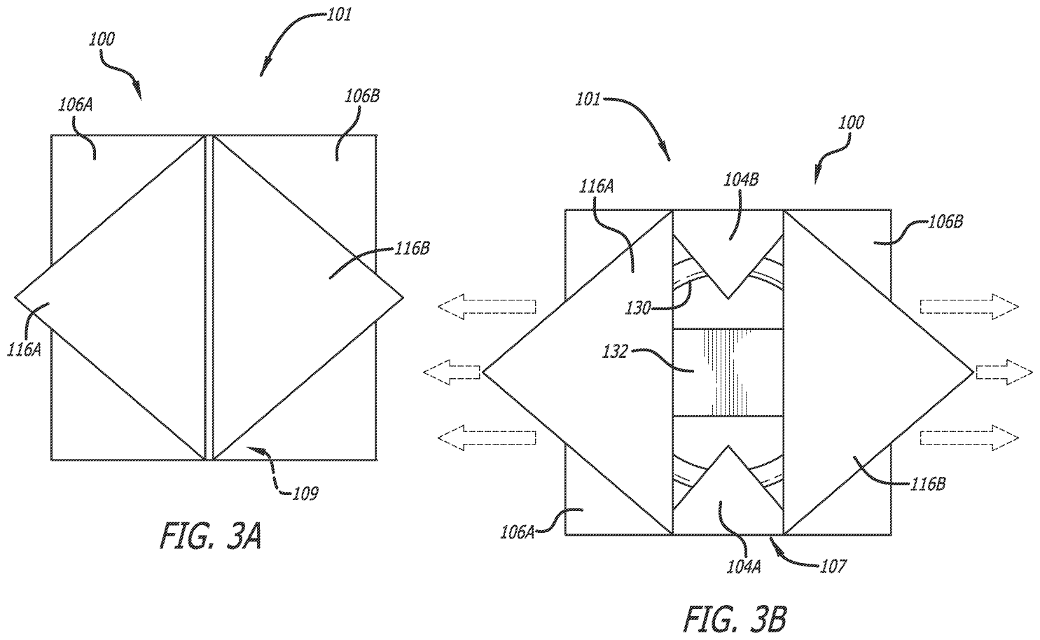

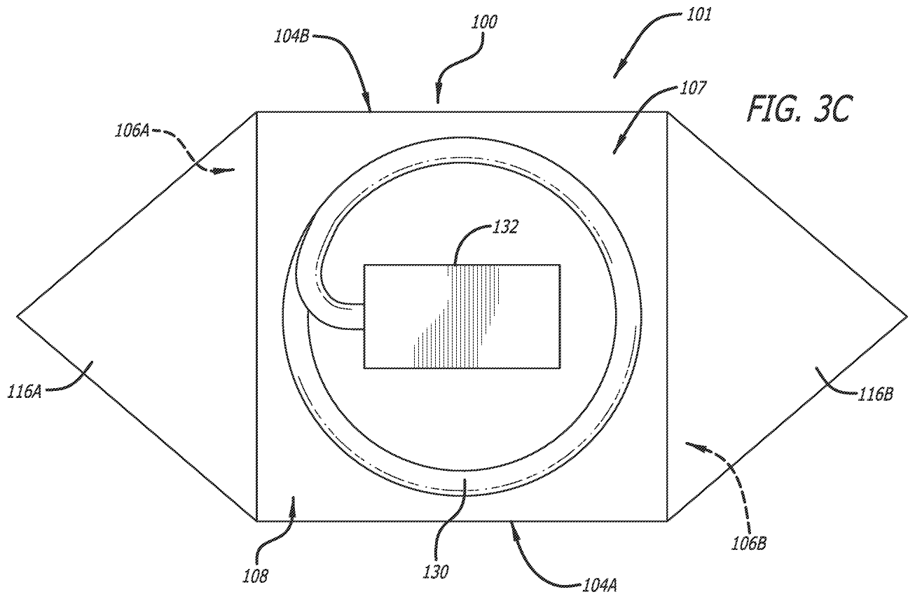

FIGS. 3A-3C illustrate a plan view of the container 100 in the closed configuration (FIG. 3A), a partially open configuration (FIG. 3B), and the fully open configuration (FIG. 3C). As illustrated in FIG. 3A, the first pull tab 116A and second pull tap 116B take the form of extensions of the first side wall 106A and the second side wall 106B, respectively. The first pull tab 116A and second pull tap 116B are sized so that at least a portion of the first pull tab 116A and second pull tap 116B extend beyond respective perimeters of the container 100 to enable the user to easily grasp the first pull tab 116A and second pull tap 116B. In some embodiments, the pull tabs 116A, 116B may be triangularly shaped so that an apex of the triangle extends beyond the perimeter. The first side wall 106A and the second side wall 106B may be pulled by the first pull tab 116A and the second pull tab 116B laterally away from each other to transition the container 100 away from the closed configuration to expose the cavity 107 as illustrated in FIG. 3B. The cavity 107 includes the catheter 130 and the fluid drainage bag 132 therein. As illustrated in FIG. 3C, the user may continue to pull the first pull tab 116A and the second pull tab 116B until the cavity 107 is completely exposed (FIG. 3C), allowing a user to reach into the cavity 107 unimpeded to retrieve the catheter 130 and the fluid drainage bag 132 from the container 100.

In some embodiments, the container 100 having the catheter 130 disposed therein may define a catheter assembly 101. In some embodiments, the catheter assembly 101 may further include the drainage bag 132 disposed within the container 100.

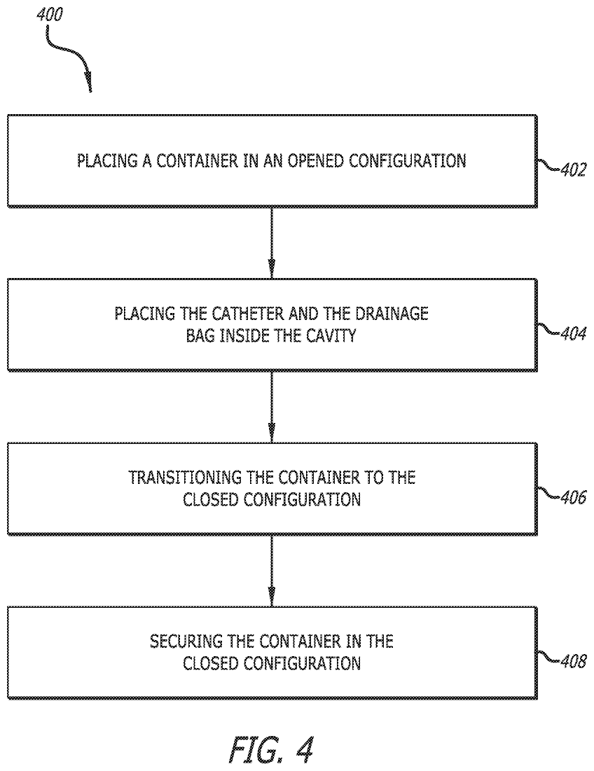

FIG. 4 illustrates a flow chart of an exemplary method 400 of packaging a catheter 130 and a fluid drainage bag 132 to define the catheter assembly 101, in accordance with some embodiments. In some embodiments, the method 400 includes disposing the container 100 in the opened configuration (block 402). The method 400 further includes placing the catheter 130 and the fluid drainage bag 132 inside the cavity 107 (block 404). In some embodiments, placing the catheter 130 includes coiling the catheter 130 before placing inside the cavity 107. The method 400 further includes transitioning the container 100 to the closed configuration (block 406). In some embodiments, the side walls 106A, 106B are configured to cover the cavity 107. The method 400 further includes securing the container 100 in the closed configuration (block 408). In some embodiments, securing the container 100 includes one or more of securing the side walls 106A, 106B together, adhering the side walls 106A, 106B to the end walls 104A, 104B, and/or sealing portions of the side walls 106A, 106B to the end walls 104A, 104B to seal the cavity 107. In some embodiments, the side walls 106A, 106B may be secured together using an adhesive or the adhesive tape 113. In some embodiments, securing the container 100 in the closed configuration includes sterilizing the catheter 130, the drainage bag 132, and container 100.

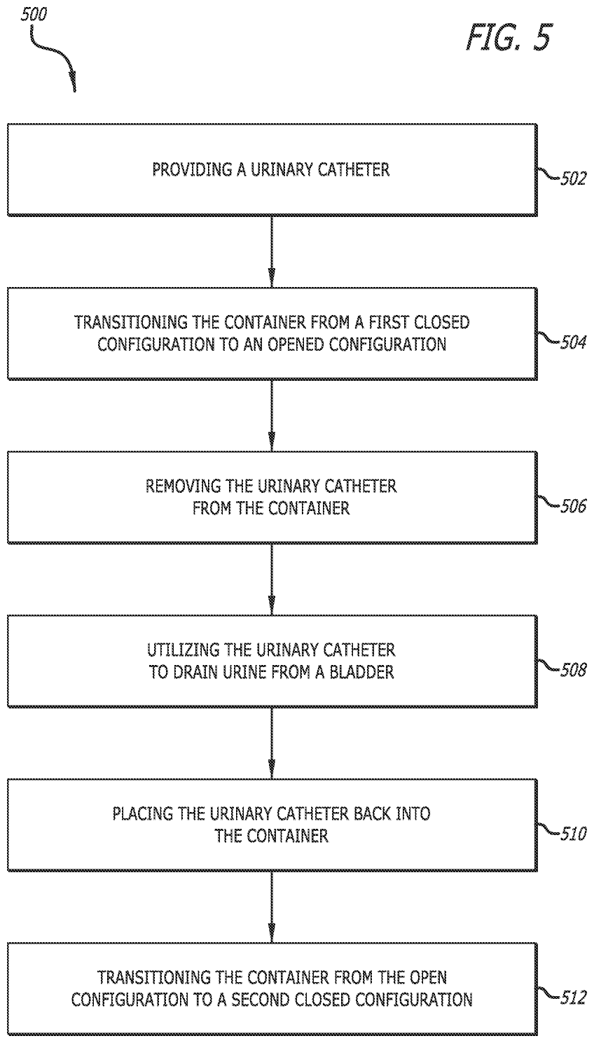

FIG. 5 illustrates a flow chart of an exemplary method 500 of draining urine from a bladder including all or a subset of the following steps or processes. One step of the method 500 includes providing the urinary catheter 130 enclosed within the container 100 in a first closed configuration of the container 100 (block 502) where the container 100 includes a bottom panel (i.e., the bottom wall 102) defining a length and a width of the container 100; first and second side panels (i.e., the side walls 106A, 106B) pivotably coupled with the bottom panel along opposite sides of the bottom panel; and first and second end panels (i.e., the end walls 104A, 104B) pivotably coupled with the bottom panel along opposite ends of the bottom panel, where each of the first and second end panels are further coupled with each of the first and second side panels. In the first closed configuration, the first and second end panels and the first and second side panels are disposed substantially parallel to the bottom panel.

Another step of the method 500 includes transitioning the container 100 from the first closed configuration to an open configuration, such that the first and second end panels and the first and second side panels are disposed substantially perpendicular to the bottom panel (block 504). In some embodiments of the method, the container 100 is biased away from the first closed configuration toward the open configuration so that when the container 100 is disposed in the open configuration, the container 100 remains in the open configuration. Transitioning the container to the open configuration may also include breaching a sterile barrier of the container 100. Transitioning the container to the open configuration may also include separating a number of adhesively bonded sections of one or more of the of the first and second end panels or first and second end panels from each other, where the adhesively bonded sections are configured to secure the container in the first closed configuration until the container 100 is transitioned to the open configuration. Transitioning the container to the open configuration may also include applying a first pulling force to the first side panel and a second pulling force to the second side panel, where the second pulling force is directed in opposition to the first pulling force. Applying the first pulling force to the first side panel and applying the second pulling force to the second side panel may include grasping a first pull tab coupled with the first side panel and grasping a second pull tab coupled with the second side panel. Transitioning the container to the open configuration may also include tearing the adhesive tape 113 extending between the first and second side panels, where the adhesive tape 113 is configured to provide indication that the container 100 has been transitioned away from the first closed configuration.

Further steps of the method include removing the urinary catheter 130 from the container 100 (block 506); utilizing the urinary catheter to drain the urine from the bladder (block 508); placing the urinary catheter 130 back into the container 100 (block 510); and transitioning the container 100 from the open configuration to a second closed configuration (block 512), such that the first and second end panels and the first and second side panels are again disposed substantially parallel to the bottom panel. Transitioning the container 100 to the second closed configuration may include recoupling one or more of the number of adhesively bonded sections together to secure the container in the second closed configuration. In some embodiments of the method, the container is configured to inhibit fluid disposed within the container from exiting the container when the container is disposed in the second closed configuration.

In some embodiments, the catheter 130 is coupled to the drainage bag 132 for containing urine therein, and as such, utilizing the catheter 130 to drain the urine from the bladder the may include collecting the urine within the drainage bag 132 and emptying the urine from the drainage bag 132 into a urine receptacle. Similarly, placing the urinary catheter 130 back into the container 100 may include placing the drainage bag 132 back into the container 100.

In some embodiments of the method, the bottom panel, the first and second side panels, and the first and second end panels each include a hydrophobic inside surface.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A medical package, comprising:
a container configured to transition from a first closed configuration to an open configuration, the container comprising:
a bottom panel defining a length and a width of the container;
first and second side panels pivotably coupled with the bottom panel along opposite sides of the bottom panel; and
first and second end panels pivotably coupled with the bottom panel along opposite ends of the bottom panel, each of the first and second end panels further coupled with each of the first and second side panels, wherein:
in the first closed configuration the first and second end panels and the first and second side panels are disposed substantially parallel to the bottom panel, and
the container is configured to enclose a urinary catheter therein in the first closed configuration.

2. The package according to claim 1, wherein in the open configuration, the first and second end panels and the first and second side panels are disposed substantially perpendicular to the bottom panel.

3. The package according to claim 1, wherein the container is biased away from the first closed configuration toward the open configuration.

4. The package according to claim 1 wherein the urinary catheter is a male urinary catheter.

5. The package according to claim 1, wherein the container is configured for handling within a clean room.

6. The package according to claim 1, wherein the container is configured to enable sterilization of the catheter when the catheter is disposed within the container, the container disposed in the first closed configuration.

7. The package according to claim 1, wherein the container defines a sterile barrier for the catheter in the first closed configuration.

8. The package according to claim 1, wherein the container includes a number of adhesively bonded sections of one or more of the first and second side panels or the first and second end panels, the adhesively bonded sections configured to secure the container in the first closed configuration.

9. The package according to claim 8, wherein the number of adhesively bonded sections are configured to define a sealed cavity for containing the urinary catheter therein in the first closed configuration.

10. The package according to claim 1, wherein the container is configured to transition from the first closed configuration to the open configuration in response to a user applying a first pulling force to the first side panel and a second pulling force to the second side panel, the second pulling force directed in opposition to the first pulling force.

11. The package according to claim 10, wherein:
the first side panel includes a first pull tab configured for grasping by the user when applying the first pulling force, and
the second side panel includes a second pull tab configured for grasping by the user when applying the second pulling force.

12. The package according to claim 11, wherein at least one of the first and second pull tabs includes a pull ring.

13. The package according to claim 11, wherein at least a portion of each of the first and second pull tabs extends outward beyond a perimeter of the container in the first closed configuration.

14. The package according to claim 1, wherein the urinary catheter includes a bag for collecting urine therein.

15. The package according to claim 14, wherein the bag is coupled with the urinary catheter when the bag is enclosed within the container, such that the bag is in fluid communication with a lumen of the urinary catheter.

16. The package according to claim 1, wherein the container is configured to:
transition from the opened configuration to a second closed configuration, and
enclose the urinary catheter within the container in the second closed configuration after use of the urinary catheter.

17. The package according to claim 16, wherein one or more of the number of adhesively bonded sections are configured to further secure the container in the second closed configuration.

18. The package according to claim 16, wherein the container is configured to inhibit fluid disposed within the container from exiting the container with the container disposed in the second closed configuration.

19. The package according to claim 1, further comprising a tamper evident mechanism configured to indicate that the container has been transitioned away from the first closed configuration.

20. The package according to claim 19, wherein the tamper evident mechanism includes a tear-able adhesive tape extending between the first and second side panels.

21. The package according to claim 1, wherein a bottom panel, the first and second side panels, and the first and second end panels each include a hydrophobic inside surface.

22. A urinary catheter assembly, comprising:
a urinary catheter; and
the package according to claim 1,
wherein the urinary catheter is enclosed within the container of the package.

23. The assembly according to claim 22, further comprising a collection bag enclosed within the container.

24. The assembly according to claim 23, wherein the collection bag is coupled with the catheter such that the bag is in fluid communication with a lumen of the urinary catheter.

25-35. (canceled)
